(19) United States
(12) Patent Application Publication
(10) Pub. No.: US 2026/0242377 A1
(43) Pub. Date: Aug. 20, 2026

(54) A PROCESS FOR THE PREPARATION OF LEDIPASVIR

(71) Applicant: COUNCIL OF SCIENTIFIC AND INDUSTRIAL RESEARCH AN INDIAN REGISTERED BODY INCORPORATED UNDER THE REGN., New Delhi (IN)

(72) Inventors: Sudhakar GANGARAJULA, Hyderabad (IN); Ramu CHENNAM, Hyderabad (IN); Thenkrishnan KUMARAGURU, Hyderabad (IN); Haridas BABURAO RODE, Hyderabad (IN); Sridhar MADDI REDDY, Hyderabad (IN); Raji CHADA REDDY, Hyderabad (IN); Subhash GHOSH, Hyderabad (IN)

(21) Appl. No.: 18/724,883

(22) PCT Filed: Nov. 24, 2023
(86) PCT No.: PCT/IN2023/051091

§ 371 Date: Jun. 27, 2024

(30) Foreign Application Priority Data
Nov. 25, 2022 (IN) 202211068208

(51) Int. Cl. C07D 471/08; C07D 207/22; C07D 403/12; C07F 5/02

(52) U.S. Cl. CPC C07D 471/08; C07D 207/22; C07D 403/12; C07F 5/025

(57) ABSTRACT

The present disclosure relates to process for the preparation of Ledipasvir (I) or of its pharmaceutically acceptable salts. The present disclosure also provides novel intermediates (IV-XI) that are used in the synthesis of Ledipasvir. The main strategies of the present invention are late-stage functionalization such as difluorination and cyclopropanation of the key intermediates of Ledipasvir (I).

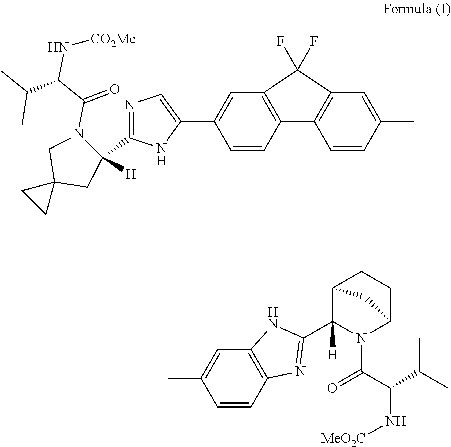

### FIELD OF THE INVENTION

The present invention relates to a process for the preparation of Ledipasvir of Formula I, its pharmaceutically acceptable salts or solvate thereof.

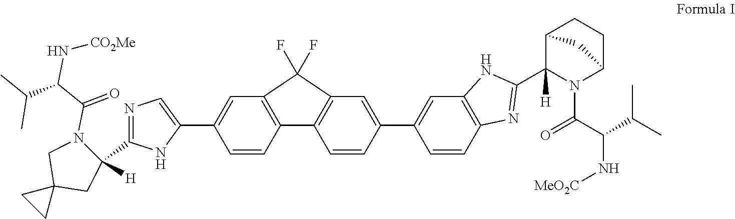 [SMILES: COC(=O)N[C@H](C(=O)N1CC2(CC2)C[C@H]1c1ncc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)[nH]1)C(C)C]

Particularly, the present invention relates to the process for the preparation of Ledipasvir by late-stage functionalization such as fluorination and cyclopropanation. The present invention further provides intermediates of compounds of formula (IV-XI) that are used in the synthesis of Ledipasvir.

### BACKGROUND OF THE INVENTION

Hepatitis C virus (HCV) causes liver infection and is the leading cause of liver transplant, jaundice, and hepatocellular carcinoma (Clin. Microbiol. Infect. 2011, 17, 107). A 2018 report from the World Health Organization revealed that approximately 71 million infected individuals globally and 399000 people die each year from this chronic blood-borne disease. Slow progression and asymptomatic for decades hamper early-stage detection and intervention, and unlike HAV and HBV, no vaccine is available for HCV treatment (https://www.who.int/en/news-room/fact-sheets/detail/hepatitis-c). The early standard of care for the treatment of HCV involving pegylated interferon-alpha and ribavirin has a low sustained virologic response (SVR), whereas the treatment regimen involving direct-acting antivirals (DAA) has high SVR. DAA agents were developed to target mainly NS3/4A protease, the NS5A protein, and the NS5B RNA-dependent RNA polymerase that are involved at different vital steps of the HCV life cycle. The HCV NS5A protein, which has no known enzymatic activity and mammalian homolog, is found to be essential for virus RNA replication, assembly, and secretion.

Ledipasvir is a direct-acting antiviral of Hepatitis C virus NS5A inhibitor developed by Gilead Sciences and is used in combination with Sofosbuvir (a nucleotide inhibitor of viral polymerase) in the fixed combination. The USFDA approved the combination of Ledipasvir and Sofosbuvir with the Harvoni Brand name and is the first combination pill to treat chronic HCV genotype 1 infection.

Structurally, Ledipasvir contains a fluorene moiety in the middle, embracing both sides with dipeptides, a combination of natural and unnatural amino acids. Generally practiced synthetic approaches for Ledipasvir involve coupling pre-functionalized fragments, fluorene and two unnatural amino acids, finally introducing valine derivatives.

The previous synthetic approaches suffer from lengthy synthetic steps, excessive amounts of expensive reagents/hazardous reagents, low-yielding steps, and difficulty handling scale-up synthesis. Notably, synthesizing of the two key intermediates such as fluorene and chiral cyclopropane are challenging.

References may be made to patent applications, WO2010132601A1, WO 2013184702 and US2013324740A1, which disclose the synthesis of fluorene intermediate from 2-bromofluorene in three steps. The second step involves the usage of an expensive fluorinating agent, and the third step, acylation using organometallic reagent and Weinreb amide (2-chloro-N-methoxy-N-methylacetamide), which is derived from chloroacetic acid. Also, the large-scale synthesis of ((S)-5-(tert-butoxycarbonyl)-5-azaspiro[2.4]heptane-6-carboxylic acid) from diethyl malonate in 6-7 steps. The enantiomeric purity of this fragment is achieved by using enzymatic resolution or chiral resolving agents from a racemic mixture of the corresponding ester. Despite the fact that enzymatic resolution provides excellent enantiomeric purity, high catalyst loading (>30% w/w) and enzyme recovery are tedious and time-consuming.

These patent applications also disclose the alternate route for the preparation of the same, commencing from Boc-4-methylene-L-Proline in three steps. However, the first step, cyclopropanation reaction on a compound of formula 1 is cumbersome, obtaining methyl ester compound of formula 2. Conversion of 2 to 6 adds an extra hydrolysis step along with the scrambling of enantiomeric purity.

Apart from this, the unreacted starting material is also transformed to its methyl ester 3 in the cyclopropanation step, posing a severe purification problem for the product. The product's purification from the unreacted starting material of formula 1 is achieved by the mixture subjecting to an iodolactonization (converting a compound of formula 1) into a separable bicyclic compound 7). The overall yield is around 45% in three steps with exhaustive operational difficulties. Alternatively, the two-step synthetic sequence; the first step delivers halogens containing cyclopropane compound 5. This was dehalogenation under a hydrogen atmosphere in the presence of a Pd/C catalyst afforded the desired compound 6 (Scheme 1).

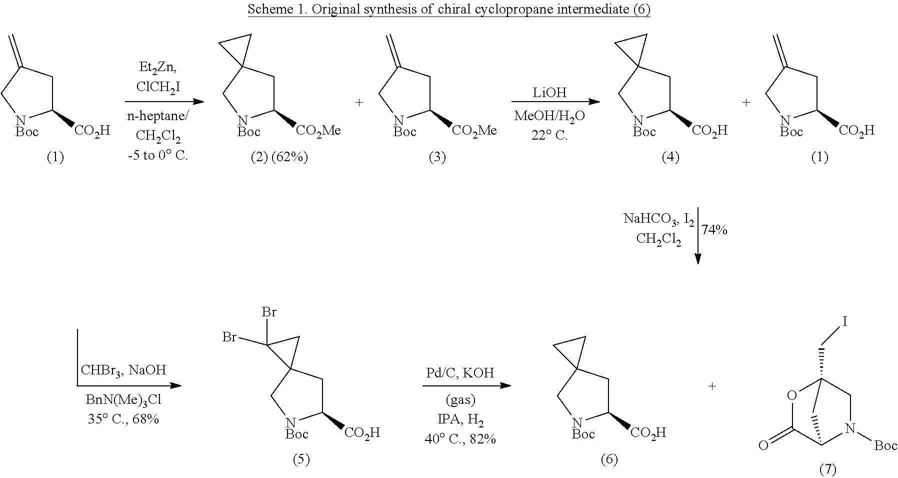

References may be made to patent applications “WO 2016/103232 A1; WO2016145990A1; WO 2018113277 A1; U.S. Pat. No. 9,056,860B2 (CN107954990); IN3237/DEL/2015; and WO2016207915A1”, wherein few more methods and intermediates for the synthesis of Ledipasvir are described and pre-functionalized fragments were used. As a result, expensive reagents, such as fluorinating reagent, is required in an excess amount as it is used early in the synthetic sequence. Further, the protection and deprotection steps are not minimized in conventional routes.

Overall, these approaches suffer from lengthy synthetic routes, low yields, and expensive/hazardous reagents, necessitating developing more efficient and shorter synthetic routes for these fluorene and chiral cyclopropane fragments.

Hence, there is a dire need in the art to develop a convenient, compatible, fast and efficient process for the preparation of chiral cyclopropane intermediates and compounds that aid in the process for preparation of

### Objects of the Invention

Main objective of the present invention is to provide a process for the preparation of Ledipasvir of Formula I, its pharmaceutically acceptable salts or solvate thereof, which involves late-stage cyclopropanation and fluorination.

Another object of the present invention is to provide intermediates of compounds of formula (IV-XI) that are used in the synthesis of Ledipasvir.

Yet another object of the present invention is to provides a process for preparing intermediates of Formula IV-XI.

### SUMMARY OF THE INVENTION

Accordingly, present invention provides a process for the preparation of Ledipasvir compound of formula I, its pharmaceutically acceptable salt or solvate thereof,

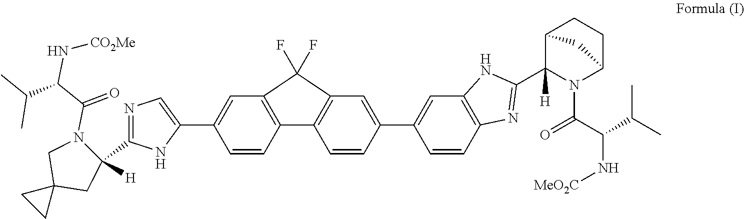 [SMILES: COC(=O)N[C@H](C(=O)N1CC2(CC2)C[C@H]1c1ncc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)[nH]1)C(C)C]

comprising the steps of:
i) coupling the compound of formula (II) or its reactive derivative with compound of formula (III) in the presence of a solvent and optionally using a catalyst to give compound of formula (IV);

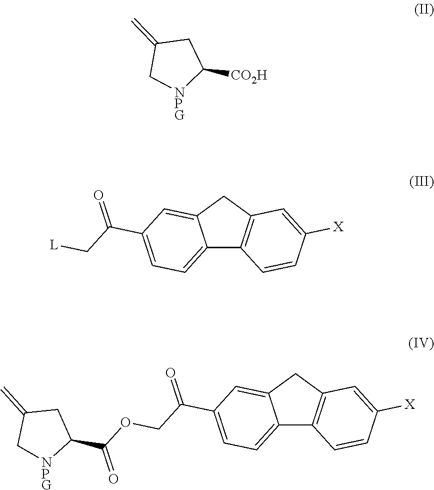

wherein
PG is a protecting group selected from the group consisting of Carbobenzyloxy (Cbz), tert-Butyloxycarbonyl (Boc), p-Methoxy carbonyl (Moz or MeOZ), 9-Fluorenylmethyloxycarbonyl (FMOC), Acetyl (Ac), Benzoyl (Bz), Benzyl carbamate, Tosyl (Ts), Sulfonamides;
L is halo or leaving group selected from the group consisting of halogens, in particular Cl, Br, F or I; alkyl boronate esters, cycloalkyl boronate esters, mesyloxy, acyloxy, tosyloxy, benzyloxy, trifluoromethylsulfonyloxy, nonafluoraobutylsulfonyloxy, (4-bromo-phenyl)sulfonyloxy, (4-nitro-phenyl)sulfonyloxy, (2-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutylphenyl)sulfonyloxy, and (4-methoxy-phenyl)sulfonyloxy preferably Cl;
X is halo or (RO)₂B—;
R is C₁₋₆ alkyl, aryl or R groups combined form together to form substituted cycloalkyl group;
ii) cyclization the compound of formula (IV) as obtained in step (i) using a cyclizing agent in a solvent to obtain compound of formula (V);

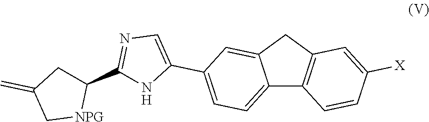

iii) protecting the compound of formula of (V) as obtained in step (ii) with a protecting group [PG] and in a solvent to give compound of formula (VI);

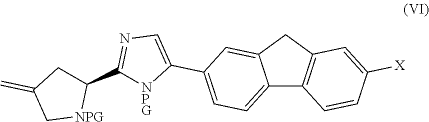

iv) cyclopropanation on the compound of Formula (VI) as obtained in step (iii) to give a compound of Formula (VII) followed by fluorination on compound of Formula (VII) to give a compound of Formula (IX) or fluorination on the compound of Formula (VI) to give a compound of Formula (VIII) and followed by cyclopropanation on compound of Formula (VIII) using a reagent and in a solvent to give a compound of Formula of (IX);

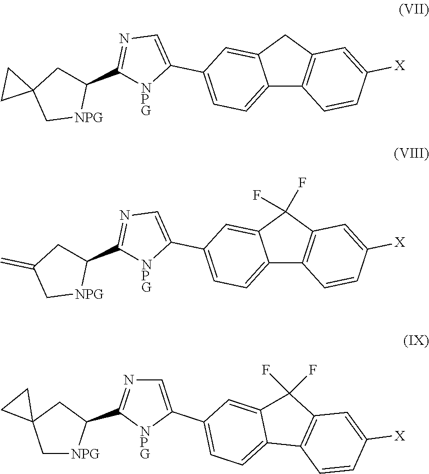

v) reacting the compound of Formula (IX) as obtained in step (iv) or its salts with the compound of Formula (IXa) using a metal catalyst and a base in the presence of a solvent to give a compound of Formula (X)

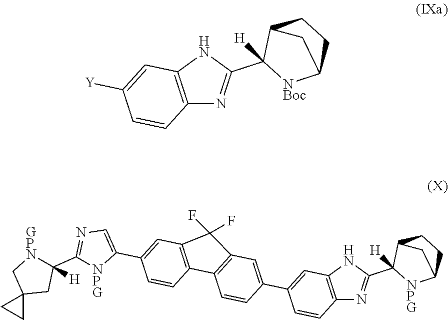

wherein
when X is halo in Formula IX, Y is —B(OR)₂ in Formula IXa;
vi) converting compound of Formula (IX) as obtained in step (iv) or its salts with boronate esters using a metal catalyst and a base in the presence of a solvent to give a compound of Formula (XI);

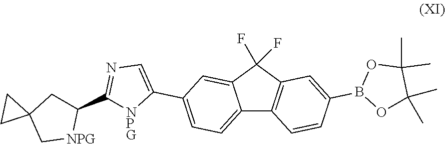

wherein
X is —B(OR)₂ in Formula IX, R is C1-6 alkyl, aryl or R groups combined form together to form substituted cycloalkyl group.
vii) alternatively, reacting the compound of Formula (XI) as obtained in step (vi) or its salts with the compound of Formula (XIa) using a metal catalyst and a base in the presence of a solvent to give a compound of Formula (X);

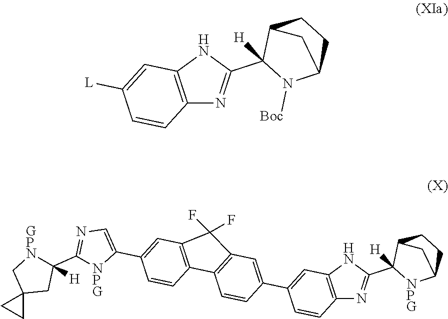

wherein Y is halo (Formula of IXa);
viii) removing protecting groups of the compound of Formula (X) as obtained in step (v) or step (vii) using an acid in a solvent followed by reaction with compound of a Formula (Xa) or its reactive derivative in the presence of a condensing agent and a solvent to give Ledipasvir of Formula I.

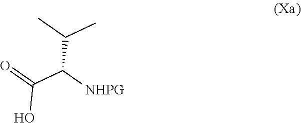

In an embodiment of the present invention, cyclizing agent is selected from the group consisting of ammonium acetate, K₂CO₃, sodium bicarbonate, sodium bicarbonate or like.

In another embodiment of the present invention, in step (iv), the cyclopropanation is carried out in the presence of sulfonium halide in presence of base, diiodomethane in presence of diethylzinc, and reagent selected from CF₃CO₂ZnCH₂I, and (PhO)₂P(O)ZnCH₂I preferably diiodomethane in the presence of diethylzinc in dichloromethane.

In yet another embodiment of the present invention, in step (iv), the fluorination is carried out using fluorinating agent selected from the group consisting of 1-chloromethyl-4-fluoro-1, 4-diazoniabicyclo [2.2.2]octanebis(tetrafluoroborate), N-fluorobenzenesulfonimide, xenon fluorides, cobalt fluoride, silver(II) fluoride, tetraethylammonium tetrafluoroborate, hydrogen fluoride pyridine complex, potassium hydrogen fluoride, triethylamine trihydrofluoride, 4-iodotoluene difluoride preferably, N-fluorobenzenesulfonimide.

In yet another embodiment of the present invention, the protecting group is selected from the group consisting of carbobenzyloxy (Cbz), tert-butyloxycarbonyl (Boc), p-methoxy carbonyl (Moz or MeOZ), 9-fluorenylmethyloxycarbonyl (FMOC), acetyl (Ac), benzoyl (Bz), benzyl carbamate, tosyl (Ts) and sulphonamides.

In yet another embodiment of the present invention, boronate ester is prepared using boronate reagent selected from the group consisting of pinacolboronates, alkyl boronates and aryl boronates.

In yet another embodiment of the present invention, metal catalyst is selected from the group consisting of Palladium(0) or (II) complexes, selected from the group consisting of tetrakis(triphenylphosphine)palladium, tris(dibenzylideneacetone)dipalladium, palladium dppf chloride, Bis(triphenylphosphine)palladium(II) acetate, Bis(triethylphosphine)palladium(II) chloride).

In yet another embodiment of the present invention, the base is selected from the group consisting of alkyl amines, ammonia (NH₃), potassium carbonate (K₂CO₃), sodium carbonate (Na₂CO₃), sodium bicarbonate (NaHCO₃), ammonium hydroxide (NH₄₀H), magnesium hydroxide (Mg(OH)₂), calcium carbonate (CaCO₃), calcium hydroxide (Ca(OH)₂), potassium hydroxide (KOH), sodium hydroxide (NaOH), sodium hydride (NaH), potassium hydride (KH), potassium tertiary butoxide (KOtBu), sodium acetate (CH₃CO₂Na), potassium acetate (CH₃CO₂K), Sodium tertiary butoxide ((CH₃)₃CONa), lithium hydroxide (LiOH), N-Methylmorpholine either alone or mixture thereof.

In yet another embodiment of the present invention, solvent used is selected from the group consisting of water, alcohol selected from the group consisting of methanol, ethanol, n-propanol, isopropanol, n-butanol and t-butanol and the like or hydrocarbon solvent selected from the group consisting of benzene, toluene, xylene, heptanes, hexane and cyclohexane and the like or ketone solvents selected from the group consisting of acetone, ethyl methyl ketone, diethyl ketone, methyl tert-butyl ketone, isopropyl ketone and the like or ester solvents selected from the group consisting of methyl acetate, ethyl acetate, propyl acetate, isopropyl acetate, n-butyl acetate, isobutyl acetate, sec-butyl acetate, and the like or nitrile solvents selected from the group consisting of acetonitrile, propionitrile, butyronitrile and isobutyronitrile and the like or ether solvents selected from the group consisting of di-tert-butylether, dimethylether, diethylether, diisopropylether, 1,4-dioxane, methyl tert-butylether, ethyl tert-butylether, tetrahydrofuran, 2-methyl tetrahydrofuran, 2-methoxyethanol and dimethoxyethane, or Amide solvents selected from the group consisting of formamide, DMF, DMAC, N-methyl-2-pyrrolidone, N-methylformamide, 2-pyrrolidone, 1-ethenyl-2-pyrrolidone preferably DCM, Toluene, acetone, DCE and THF.

In yet another embodiment of the present invention, condensing agent used is selected from the group consisting of Benzotriazol-1-yl)-N,N,N,N-Hydroxy benzotriazole (HOBt), 0-Tetramethyluronium fluorophosphate (HBTU), 0-(Benzotriazol-1-yl)-N,N,N,N-tetramethyluroniumtetra fluoroborate (TBTU), 1-Hydroxy-7-azabenzotriazole (HOAt), N,N′-Dicyclohexyl carbodiimide (DCC), N-(3-Dimethylaminopropyl)-N′-ethyl carbodiimide hydrochloride (EDC-HCl), N,N′-Carbonyl diimidazole (CDI), Benzotriazol-1 yloxytris (dimethyl amino)phosphonium hexafluorophosphate (BOP), Benzotriazolyloxytripyrrolidinophosphonium hexafluorophosphate (PyBOP), and 1[Bis(dimethylamino)methylene]-1H-1,2,3-triazole[4,5-b]pyridinium 3-oxide hexafluorophosphate (HATU) either alone or mixture thereof.

In yet another embodiment, present invention provides a compound of formula A

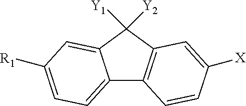

### Wherein R1=

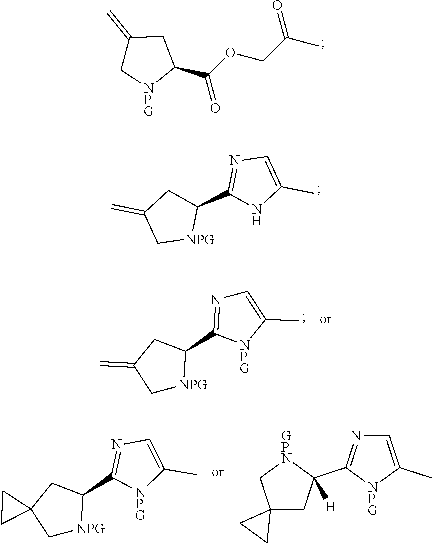

Y₁ and Y₂=H or F;

X is halo or (RO)₂B—;

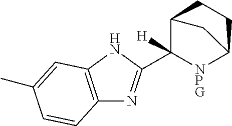

In yet another embodiment of the present invention, compound of formula A is selected from the group consisting of:

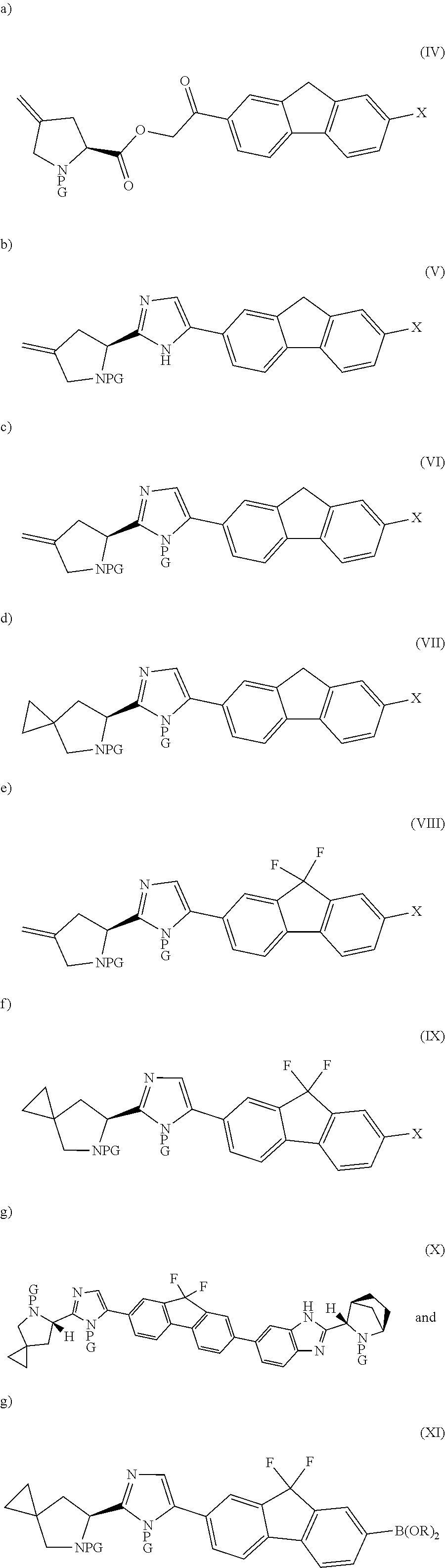

### BRIEF DESCRIPTION OF THE DRAWINGS

The following drawings form a part of the present specification and are included to further illustrate aspects of the present disclosure. The disclosure may be better understood by reference to the drawings in combination with the detailed description of the specific embodiments presented herein.

FIG. 1 represents general process steps for the preparation of Ledipasvir of Formula (I), in accordance with an embodiment of the present disclosure.

FIG. 2 represents process steps for the preparation of Ledipasvir of Formula (I), in accordance with an embodiment of the present disclosure.

FIG. 3 represents an alternate process for the preparation of a compound of formula (IX), in accordance with an embodiment of the present disclosure.

FIG. 4 represents an alternate process for the preparation of a compound of formula (X), in accordance with an embodiment of the present disclosure.

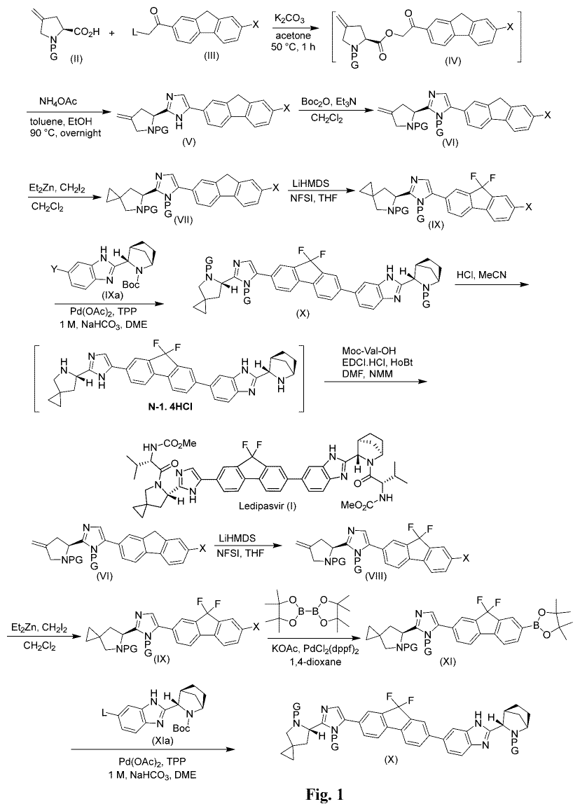

### DETAIL DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in light of the remainder of the disclosure and understood as by a person of skill in the art. The terms used herein have the meanings recognized and known to those of skill in the art, however, for convenience and completeness, particular terms and their meanings are set forth below.

The term “at least one” is used to mean one or more and thus includes individual components as well as mixtures/combinations.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “including” and “including but not limited to” are used interchangeably.

Unless the context requires otherwise, throughout the specification which follow, the word “comprise” and variations thereof, such as, “comprises” and “comprising” are to be construed in an open, inclusive sense that is as “including, but not limited to.”

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it is individually recited herein.

All processes described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Various terms are used herein. To the extent a term used in a claim is not defined below, it should be given the broadest definition persons in the pertinent art have given that term as reflected in printed publications and issued patents at the time of filing.

As used herein, the term “cyclopropanation” refers to the process of reacting a compound with a reagent to result in cyclopropane entity in a portion of the compound or result in conversion of the compound to cyclopropane. In an aspect of the present disclosure, the cyclopropanation is carried out in the presence of a sulfonium halide in the presence of a base, preferably diiodomethane in the presence of diethylzinc in dichloromethane.

As used herein, the term “fluorination” refers to the process of substituting an atom of a compound with fluorine atom. In an aspect of the present disclosure, the fluorination is carried out using a fluorinating agent selected from the group consisting of 1-chloromethyl-4-fluoro-1, 4-diazoniabicyclo [2.2.2]octanebis(tetrafluoroborate), N-fluorobenzenesulfonimide, xenon fluorides, cobalt fluoride, silver(II) fluoride, tetraethylammonium tetrafluoroborate, hydrogen fluoride pyridine complex, potassium hydrogen fluoride, triethylamine trihydrofluoride, or 4-iodotoluene difluoride and preferably, N-fluorobenzenesulfonimide.

As discussed in the background, there occur many undesirable transformations of compounds, requirement of high amounts of costly reagents, complex reaction steps and time consuming for the conventional process for preparation of Ledipasvir, wherein the cyclopropanation and fluorination is carried out at the prior stages.

The present invention discloses the usage of late-stage cyclopropanation and fluorination, which would substantially reduce the several unwanted transformations, and the amount of fluorinating reagent, N-fluorobenzenesulfonimide (NFSI), required at the beginning. None of the above cited literatures have suggested the yield improvement through late stage cylcopropanation and fluorination, which leads to improvement in overall yield.

The present invention provides a process for the preparation of Ledipasvir, its pharmaceutically acceptable salts or solvate thereof.

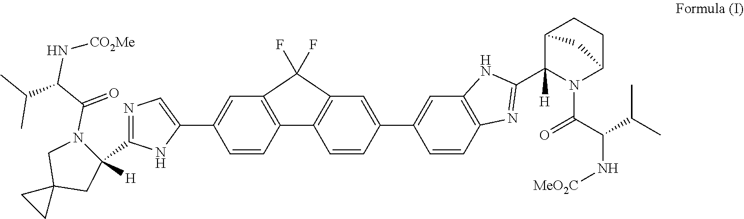 [SMILES: COC(=O)N[C@H](C(=O)N1CC2(CC2)C[C@H]1c1ncc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)[nH]1)C(C)C]

The compounds of Formula IV, V, VI, VII, VIII, IX, X, and XI or their salts are used in the present invention. Any of the above reactions may be carried out in-situ reactions to obtain Ledipasvir or its salts. The above compounds may be isolated as salts or free bases, and if the above compounds are isolated as salts, they are converted to their free bases first and used for further reactions. Further, the above compound may be isolated as crystalline forms or isolated as an amorphous form or optionally recrystallized and used for further reactions.

The process for the preparation of Ledipasvir of Formula I, its pharmaceutically acceptable salts or solvate thereof comprising the steps of:
i. coupling the compound of Formula (II) or its reactive derivative with a compound of Formula (III) in the presence of a solvent and optionally using a catalyst to give a compound of Formula (IV);

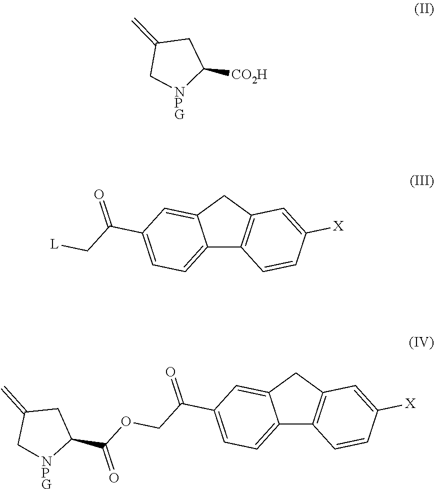

wherein
PG represents a protecting group;
L is halo or leaving group;
X is halo or (RO)₂B—
R is C₁₋₆ alkyl, aryl or
R groups combined form together to form substituted cycloalkyl group;
ii. cyclizing compound of Formula (IV) as obtained in step (i) using a cyclizing agent in a solvent to obtain a compound of Formula (V);

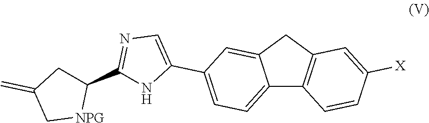

iii. protecting of a compound of Formula of (V) as obtained in step (ii) with a protecting group [PG] and in a solvent to give a compound of Formula (VI);

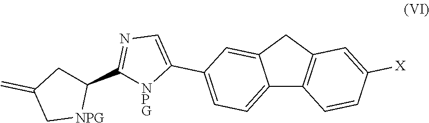

iv. cyclopropanation on the compound of Formula (VI) as obtained in step (iii) to give a compound of Formula (VII) followed by fluorination on compound of Formula (VII) to give a compound of Formula (IX) or fluorination on the compound of Formula (VI) to give a compound of Formula (VIII) and followed by cyclopropanation on compound of Formula (VIII) using a reagent and in a solvent to give a compound of Formula of (IX);

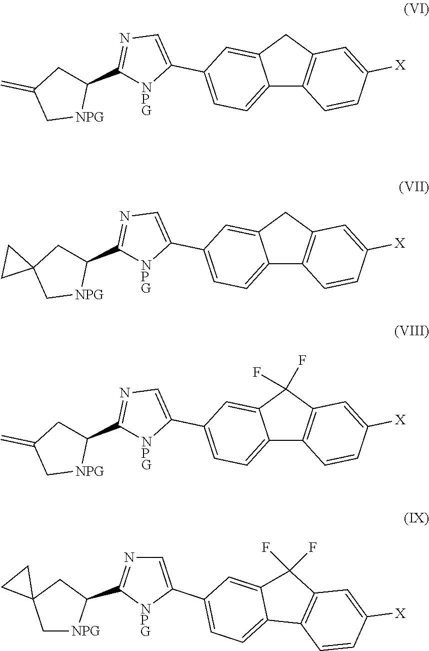

v. reacting the compound of Formula (IX) as obtained in step (iv) or its salts with the compound of Formula (IXa) using a metal catalyst and a base in the presence of a solvent to give a compound of Formula (X);

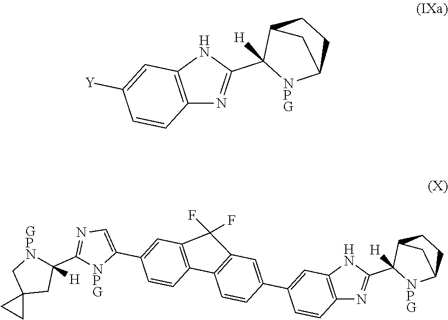

Wherein
when X=halo (Formula of IX), Y is —B(OR)₂ (Formula of IXa);
vi. converting compound of Formula (IX) as obtained in step (iv) or its salts with boronate esters using a metal catalyst and a base in the presence of a solvent to give a compound of Formula (XI);
where X=—B(OR)₂ (Formula of IX), R is C1-6 alkyl, aryl or R groups combined form together to form substituted cycloalkyl group;
vii. alternatively, reacting the compound of Formula (XI) where X=B(OR)₂ as obtained in step (vi) or its salts with the compound of Formula (XIa), wherein Y is halo (Formula of IXa); using a metal catalyst and a base in the presence of a solvent to give a compound of Formula (X);
viii. removing protecting groups of the compound of Formula (X) as obtained in step (v) or step (vii) using an acid reagent in a solvent followed by reaction with compound of a Formula (Xa) or its reactive derivative in the presence of a condensing agent and a solvent to give Ledipasvir of Formula I;

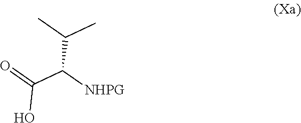

The compound of Formula (II) is coupled with a compound of Formula (III) employing in the presence of a base yielding compound of Formula (IV). The compound of Formula (IV) is cyclized in the presence of suitable reagents in a solvent to give a compound of Formula (V). The compound of Formula (V) is protected with protecting groups mentioned above to provide a compound of Formula (VI). This compound of Formula (VI) is subjected to cyclopropanation in the presence of a suitable reagent in a solvent to give a compound of Formula (VII). The compound of Formula (VII) is fluorinated in the presence of a suitable reagent in a solvent to give a compound of Formula (IX). The compound of Formula (IX) is coupled with a compound of Formula (IXa) employing metal catalyst in a solvent in the presence of a base yielding compound of Formula (X). The protecting groups on compound Formula (X) are removed, followed by peptide coupling of compound Formula (Xa) in the presence of condensing agent to yield Ledipasvir of Formula (I) in good yield.

Alternatively, the compound of Formula (VI) is difluorinated first in the presence of a reagent in a solvent to give a compound of Formula (VIII). This compound of Formula (VIII) is subjected to cyclopropanation in the presence of a suitable reagent in a solvent to give a compound of Formula (IX).

Alternatively, the compound of Formula (IX) is converted to boronate ester in the presence of a reagent in a solvent to give a compound of Formula (XI). This compound of Formula (XI) is coupled with a compound of Formula (XIa) employing metal catalyst in a solvent in the presence of a base yielding compound of Formula (X).

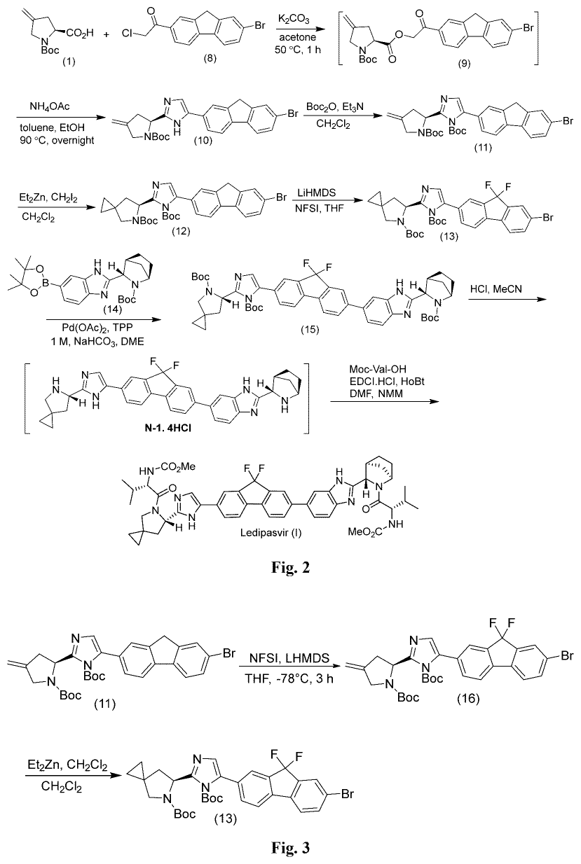

Alternatively, the compound of Formula 15 is prepared from the compound of Formula 11. The compound of Formula 11 was converted to a difluorinated compound of Formula 16. This compound of Formula 16 was converted to the cyclopropane derivative compound of Formula 13. Then, the compound of Formula 13 was coupled with the compound of Formula 14 as above to obtain a compound of Formula 15. The preparation of a compound of Formula 15 is depicted in FIG. 2.

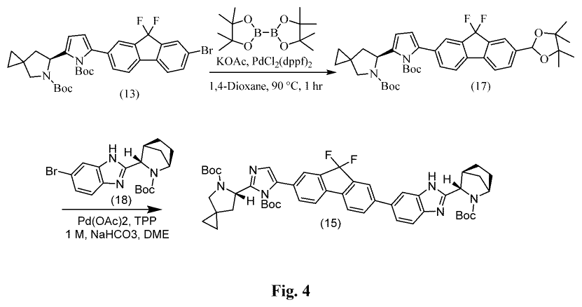

Alternatively, the compound of Formula 15 is prepared from the compound of Formula 13. The compound of Formula 13 is converted to the boronate derivative of a compound of Formula 17. The coupling of a compound of Formula of 17 is then coupled with a compound of Formula 18 to yield a compound of Formula 15. The preparation of a compound of Formula 15 is depicted in FIG. 4.

The leaving groups L is selected from the group consisting of halogens, in particular Cl, Br, F or I; alkyl boronate esters, cycloalkyl boronate esters, mesyloxy, acyloxy, tosyloxy, benzyloxy, trifluoromethylsulfonyloxy, nonafluoraobutylsulfonyloxy, (4-bromo-phenyl)sulfonyloxy, (4-nitro-phenyl)sulfonyloxy, (2-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutylphenyl)sulfonyloxy, and (4-methoxy-phenyl)sulfonyloxy preferably Cl.

The protecting group is selected from the group consisting of Carbobenzyloxy (Cbz), tert-Butyloxycarbonyl (Boc), p-Methoxy carbonyl (Moz or MeOZ), 9-Fluorenylmethyloxycarbonyl (FMOC), Acetyl (Ac), Benzoyl (Bz), Benzyl carbamate, Tosyl (Ts), Sulfonamides.

“Solvent” as defined in the present invention is selected from water or “alcohol solvents” such as methanol, ethanol, n-propanol, isopropanol, n-butanol and t-butanol and the like or “hydrocarbon solvents” such as benzene, toluene, xylene, heptanes, hexane and cyclohexane and the like or “ketone solvents” such as acetone, ethyl methyl ketone, diethyl ketone, methyl tert-butyl ketone, isopropyl ketone and the like or “ester solvents” such as methyl acetate, ethyl acetate, propyl acetate, isopropyl acetate, n-butyl acetate, isobutyl acetate, sec-butyl acetate, and the like or “nitrile solvents” such as acetonitrile, propionitrile, butyronitrile and isobutyronitrile and the like or “ether solvents” such as di-tert-butylether, dimethylether, diethylether, diisopropylether, 1,4-dioxane, methyl tert-butylether, ethyl tert-butylether, tetrahydrofuran, 2-methyl tetrahydrofuran, 2-methoxyethanol and dimethoxyethane, or “Amide solvents” such as formamide, DMF, DMAC, N-methyl-2-pyrrolidone, N-methylformamide, 2-pyrrolidone, 1-ethenyl-2-pyrrolidone and/or mixtures thereof.

“Base” as defined in the present invention is selected from alkyl amines, NH₃, K₂CO₃, Na₂CO₃, NaHCO₃, NH₄OH, Mg(OH)₂, CaCO₃, Ca(OH)₂, KOH, NaOH, NaH, KH, KOtBu, CH₃CO₂Na, CH₃CO₂K, (CH₃)₃CONa, LiOH, N-Methylmorpholine and/or mixture thereof.

Condensing agent as defined in the present invention is selected from Benzotriazol-1-yl)-N,N,N,N-Hydroxy benzotriazole (HOBt), O-Tetramethyluronium fluorophosphate (HBTU), O-(Benzotriazol-1-yl)-N,N,N,N′-tetramethyluroniumtetra fluoroborate (TBTU), 1-Hydroxy-7-azabenzotriazole (HOAt), N,N′-Dicyclohexyl carbodiimide (DCC), N-(3-Dimethylaminopropyl)-N′-ethyl carbodiimide hydrochloride (EDC-HCl), N,N′-Carbonyl diimidazole (CDI), Benzotriazol-1 yloxytris (dimethyl amino)phosphonium hexafluorophosphate (BOP), Benzotriazolyloxytripyrrolidinophosphonium hexafluorophosphate (PyBOP), and 1[Bis(dimethylamino)methylene]-1H-1,2,3-triazole[4,5-b]pyridinium 3-oxide hexafluorophosphate (HATU) or and/or mixture thereof.

Metal catalyst as defined in the present invention is selected from Palladium(0) or (II) complexes, selected from tetrakis(triphenylphosphine)palladium, tris(dibenzylideneacetone)dipalladium, palladium dppf chloride, Bis(triphenylphosphine)palladium(II) acetate, Bis(triethylphosphine)palladium(II) chloride.

Cyclization as defined in the present invention is carried out in the presence of mild basic conditions such as ammonium acetate, K₂CO₃, sodium bicarbonate, sodium carbonate, etc in presence of suitable solvent.

Deprotection as defined in the present invention is carried out in the presence of metal catalyst, hydrogen source, wherein the metal catalyst is selected from Pd, or the deprotection may be carried out in the presence of an acid which is selected from strong acids such as HCl or CF₃CO₂H or the deprotection may be carried out in the presence of a base, which is selected from primary or secondary amines.

“Boronate ester or its derivatives” as defined in the present invention is prepared using Boronate reagent, which is selected from pinacolboronates, alkyl boronates and aryl boronates.

“Acid or acidic condition” as defined in the present invention is selected from hydrochloric, hydrobromic, sulfuric, phosphoric, oxalic, maleic, succinic, citric, acetic and p-toluenesulfonic acid.

### EXAMPLES

The following examples are given by way of illustration and therefore should not be construed to limit the scope of the invention.

### Example 1: (S)-2-(2-(7-bromo-9H-fluoren-2-yl)-2-oxoethyl) 1-tert-butyl 4-methylenepyrrolidine-1,2-dicarboxylate (9); (compound of Formula (IV))

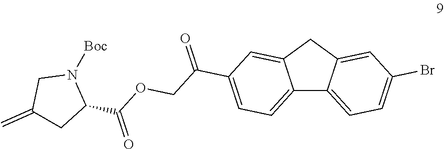 [SMILES: C=C1C[C@@H](C(=O)OCC(=O)c2ccc3c(c2)Cc2cc(Br)ccc2-3)N(C(=O)OC(C)(C)C)C1]

To a stirred solution of the compound of Formula (1) (5 g) in anhydrous acetone (60 mL) potassium carbonate (4.55 g) was added under argon and stirred for 30 minutes at 27-30° C. After that, a solution of the compound of Formula (8) (7.092 g) in acetone (30 mL) was added to the reaction mixture and stirred for 1 h at 55° C. The reaction mixture was concentrated under reduced pressure, and the resultant residue was diluted with EtOAc, washed with water, brine solution, and dried over anhydrous sodium sulfate. The solvents were removed under reduced pressure to give the product of Formula (9) (9.5 g, 81%). The synthesis procedure is depicted in FIG. 2.

¹H NMR (400 MHz, CDCl₃): δ=8.09-8.04 (m, 1H), 7.93 (d, J=7.95 Hz, 1H), 7.80 (d, J=7.95 Hz, 1H), 7.73 (s, 1H), 7.69 (d, J=8.15 Hz, 1H), 7.55 (d, J=8.15 Hz, 1H), 5.63 (d, J=16.27 Hz, 0.5H), 5.47 (d, J=16.10 Hz, 0.5H), 5.36 (d, J=16.10 Hz, 0.5H), 5.23 (d, J=16.27 Hz, 0.5H), 5.11-5.00 (m, 2H), rotamer; 4.66 (dd, J=4.66, 4.08 Hz, 0.5H), 4.60 (dd, J=9.36, 3.35 Hz, 0.5H), 4.17-4.07 (m, 2H), 3.94 (s, 2H), 3.15-2.94 (m, 2H), 1.48 (s, 4H), 1.47 (s, 5H) ppm; ¹³C NMR (75 MHz, CDCl₃): δ=191.62, 191.17, 172.19, 172.09, 154.49, 153.79, 146.34, 146.15, 143.34, 143.17, 142.36, 139.23, 132.77, 130.43, 128.58, 127.21, 124.48, 122.42, 122.21, 120.07, 108.19, 108.04, 80.40, 80.30, 66.25, 66.16, 59.00, 58.56, 50.99, 50.66, 36.85, 36.73, 36.05, 28.43, 28.35. ppm. IR (neat): νₘₐₓ 2975, 2931, 1754, 1697, 1610, 1397, 1366, 1250, 1174, 1117, 1060, 1006, 979, 897, 855, 812, 771 cm⁻¹. HRMS (ESI) m/z calculated for C₂₆H₂₇BrNO₅Br [M+H]⁺: clcd 512.1080, fund 512.1073.

### Example 2: (S)-tert-butyl 2-(5-(7-bromo-9H-fluoren-2-yl)-1H-imidazol-2-yl)-4 ethylenepyrrolidine-1-carboxylate (10); (compound of Formula (V))

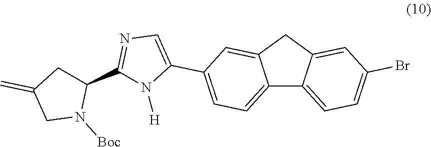 [SMILES: C=C1C[C@@H](c2ncc(-c3ccc4c(c3)Cc3cc(Br)ccc3-4)[nH]2)N(C(=O)OC(C)(C)C)C1]

To a stirred solution of the compound of Formula (9) (3 g) in anhydrous toluene (18 mL) under nitrogen atmosphere, ammonium acetate (2.25 g) and a catalytic amount of ethanol (2 mL) were added. The resulting solution was stirred for 10 h at 90° C. The reaction was quenched with a saturated aqueous NH₄Cl solution and diluted with EtOAc. The separated organic layer was washed with brine solution, dried over anhydrous sodium sulfate, and concentrated under reduced pressure to give compound of Formula (10) (2.4 g, 83%).

¹H NMR (400 MHz, DMSO-d₆): δ=11.97-11.79 (br, m, 1H), 7.95 (s, 1H), 7.91-7.73 (m, 4H), 7.61-7.51 (br, m, 2H), 5.02 (d, J=16.34 Hz, 3H), 4.5-4.23 (m, 2H), 3.96 (s, 2H), 3.14-2.98 (m, 1H), 2.71-2.57 (m, 1H), 1.47 (s, 4H), 1.21 (s, 5H) ppm; ¹³C NMR (75 MHz, DMSO-d₆): δ=153.90, 150.34, 146.04, 144.13, 143.73, 141.04, 140.28, 138.31, 134.80, 130.09, 128.54, 123.64, 121.88, 121.22, 120.74, 119.66, 112.80, 107.97, 79.66, 55.78, 51.18, 38.86, 36.83, 28.41. ppm. IR (neat): νₘₐₓ 3420, 2890, 2252, 2126, 1651, 1050, 1023, 1004, 821, 663. cm⁻¹. HRMS(ESI) m/z calculated for C₂₆H₂₇O₂N₃Br [M+H]⁺:492.128662, found: 492.12812.

### Example 3: (S)-tert-butyl 5-(7-bromo-9H-fluoren-2-yl)-2-(1-(tert-butoxycarbonyl)-4-methylenepyrrolidin-2-yl)-1H-imidazole-1-carboxylate (11); (compound of Formula (VI))

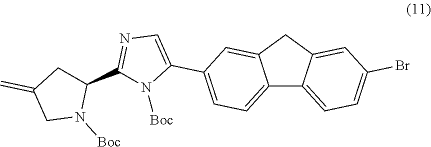 [SMILES: C=C1C[C@@H](c2ncc(-c3ccc4c(c3)Cc3cc(Br)ccc3-4)n2C(=O)OC(C)(C)C)N(C(=O)OC(C)(C)C)C1]

To a 50 mL round-bottomed flask were added compound of Formula (10) (2 g), triethylamine (1.24 mL), 4-(dimethylamino) pyridine (0.099 g), anhydrous dichloromethane (DCM) (12 mL), and a stir bar. The solution was stirred at rt, and to this was added di-tert-butyl dicarbonate (1.39 mL). The progress of the reaction was monitored by thin layer chromatography (TLC). Once the reaction was completed, the solution was transferred to a separating funnel, and H₂O (5 mL) was added. The layers were separated, the organic layer was washed with water, brine solution, dried over anhydrous sodium sulfate, and concentrated under reduced pressure to give the product of Formula (11) (2.2 g, 91.6%).

¹H NMR (500 MHz, CDCl₃): δ=7.99 (s, 0.5H), 7.92 (s, 0.5H), 7.80-7.75 (m, 1H), 7.72-7.64 (m, 2H), 7.62-7.55 (m, 2H), 7.48 (dd, J=8.12, 1.61 Hz, 1H), rotamer; 5.64 (d, J=8.35, 0.5 Hz), 5.58 (dd, J=8.67, 2.19 Hz, 0.5H), 5.07-4.90 (m, 2H), 4.49-4.38 (m, 1H), 4.27-4.18 (m, 1H), 3.89 (s, 2H), 3.21-3.10 (m, 1H), 2.64 (dd, J=31.58, 16.01 Hz, 1H), 1.66 (s, 5H), 1.64 (s, 4H), 1.51 (s, 4H), 1.25 (s, 5H) ppm; ¹³C NMR (100 MHz, CDCl₃): δ=154.74, 154.42, 152.17, 150.80, 147.77, 147.68, 145.52, 145.46, 144.67, 143.62, 143.37, 143.18, 140.71, 140.60, 140.10, 140.03, 139.91, 139.80, 132.57, 132.06, 129.90, 128.27, 124.23, 121.96, 121.04, 120.36, 120.25, 119.98, 119.94, 113.50, 112.70, 107.66, 107.45, 85.64, 85.51, 79.70, 79.42, 56.18, 55.96, 51.45, 51.27, 39.16, 38.84, 36.82, 28.62, 28.23, 28.02 ppm; IR (neat): νₘₐₓ 2977, 2931, 1750, 1699, 1458, 1391, 1367, 1288, 1255, 1160, 1104, 1060, 897, 847, 814, 758 cm⁻¹. HRMS (ESI) m/z calculated for C₃₁H₃₄BrN₃O₄ [M+H]⁺: Calcd 592.18147, found 592.18055.

### Example 4: tert-butyl 6-(5-(7-bromo-9H-fluoren-2-yl)-1-(tert-butoxycarbonyl)-1H-imidazol-2-yl)-5-azaspiro[2.4]heptane-5-carboxylate (12); (compound of Formula (VII))

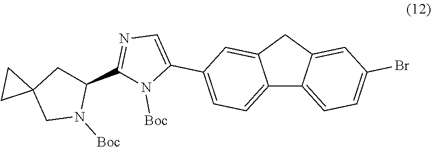 [SMILES: CC(C)(C)OC(=O)N1CC2(CC2)C[C@H]1c1ncc(-c2ccc3c(c2)Cc2cc(Br)ccc2-3)n1C(=O)OC(C)(C)C]

Diethylzinc (1M in hexane, 20.27 mL) was added to a stirred solution of the compound of Formula (11) (2 g) in anhydrous DCM at 0° C. Diiodomethane (3.27 mL) was added slowly, keeping the internal temperature below 5° C. After the addition was completed, the mixture was stirred at 0° C. for 1 hr. The reaction was quenched with a saturated aqueous NH₄Cl solution and water. Separated the layers, and the aqueous layer was extracted with DCM. The combined organic layers were dried over anhydrous sodium sulfate and concentrated under reduced pressure to give the product of Formula (12) (1.8 g, 87.8%).

¹H NMR (400 MHz, CDCl₃): δ=8.02 (s, 0.5H), 7.95 (s, 0.5H), 7.82 (d, J=8.07 Hz, 1H), 7.72 (d, J=7.98 Hz, 1H), 7.67 (d, J=4.33 Hz, 1H), 7.65-7.59 (m, 1H) 7.57 (s, 1H), 7.49 (d, J=8.07 Hz, 1H), 5.67-5.57 (m, 1H), 3.91 (s, 2H), 3.78 (d, J=10.16 Hz, 0.5H), 3.58 (dd, J=29.07, 10.01 Hz, 1H), 3.46 (d, J=10.10 Hz, 0.5H), 2.52 (dd, J=12.48, 7.92 Hz, 0.5H), 2.33 (dd, J=12.48, 7.91 Hz, 0.5H), 2.10-1.99 (m, 1H), 1.65 (s, 6H), 1.62 (s, 3H), 1.49 (s, 4H), 1.22 (S, 5H), 0.77-0.42 (m, 4H) ppm; ¹³C NMR (100 MHz, CDCl₃): δ=154.14, 152.97, 151.48, 147.81, 147.73, 145.52, 145.47, 143.52, 143.40, 143.18, 140.74, 140.62, 140.01, 139.90, 139.74, 132.24, 129.92, 128.28, 124.37, 124.27, 122.00, 121.06, 120.23, 120.02, 119.93, 113.30, 112.43, 85.47, 85.30, 79.38, 79.10, 56.66, 56.06, 54.76, 54.25, 41.43, 40.86, 36.83, 28.63, 28.22, 28.01, 20.06, 13.92, 11.20, 10.62, 8.47 ppm. IR (neat): νₘₐₓ 2976, 2930, 2869, 1750, 1697, 1458, 1394, 1368, 1350, 1287, 1257, 1163, 1106, 1060, 848, 814, 762 cm⁻¹. HRMS (ESI) m/z calculated for C₃₂H₃₇BrN₃O₄ [M+H]⁺: clcd 606.1967, found 606.1981.

### Example 5: (R)-tert-butyl 6-(5-(7-bromo-9,9-difluoro-9H-fluoren-2-yl)-1-(tert-butoxycarbonyl)-1H-imidazol-2-yl)-5-azaspiro[2.4]heptane-5-carboxylate (13); (compound of Formula (IX))

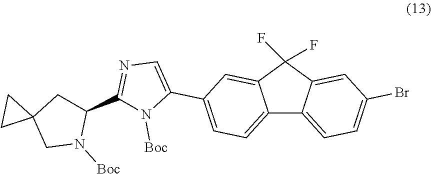 [SMILES: CC(C)(C)OC(=O)N1CC2(CC2)C[C@H]1c1ncc(-c2ccc3c(c2)C(F)(F)c2cc(Br)ccc2-3)n1C(=O)OC(C)(C)C]

The compound of Formula (12) (1 g) and N-fluorobenzenesulfonimide (1.56 g), and tetrahydrofuran (10 mL) were taken into the round bottom flask. The solution was degassed three times by slowly applying a vacuum followed by breaking vacuum with nitrogen. The solution was cooled to −78° C. A solution of the base (1.0 M LiHMDS in THF 4.9 mL) was added at such a rate the internal temperature was kept below −55° C. The reaction completion was monitored by TLC. After completion of the reaction, it was quenched by the addition of methanol. After the internal temperature had warmed to −20° C. and 0° C., hexane was added, and the solution was stirred for 10 minutes. The slurry was filtered, and the solids were rinsed with EtOAc twice. The filtrate was concentrated under reduced pressure to give the product of Formula (13) (0.82 g, 82%). The synthesis procedure is depicted in FIG. 3.

¹H NMR (125 MHz, CDCl₃): δ=8.05-8.01 (m, 1H), 7.98-7.89 (m, 1H), 7.75 (s, 1H), 7.62-7.51 (m, 3H), 7.45-7.40 (m, 1H), rotamer; 5.60 (d, J=7.82 Hz, 0.5H), 5.59 (d, J=7.82 Hz, 0.5H), 3.75 (d, J=10.16 Hz, 0.5H), 3.60 (d, J=10.16 Hz, 0.5H), 3.55 (d, J=10.16 Hz, 0.5H), 3.47 (d, J=10.16 Hz, 0.5H), 2.53 (dd, J=12.68, 7.99 Hz, 0.5H), 2.35 (dd, J=12.68, 7.99 Hz, 0.5H), 2.00 (dd, J=12.68, 7.99 Hz, 1H), 1.65 (s, 5H), 1.62 (s, 4H), 1.49 (s, 4H), 1.22 (S, 5H), 0.73-0.45 (m, 4H) ppm. ¹⁹F NMR (400 MHz, CDCl₃): −110.59 (d), −110.84 (d); ¹³C NMR (100 MHz, CDCl₃): δ=154.53, 154.04, 153.30, 151.78, 147.64, 147.56, 140.15, 139.89, 139.63, 139.04, 138.75, 138.46, 138.42, 138.38, 137.98, 137.91, 137.30, 137.00, 135.06, 134.04, 134.45, 129.32, 128.99, 127.28, 122.42, 122.10, 121.99, 121.69, 121.60, 120.86, 120.80, 120.55, 120.47, 114.02, 113.23, 85.77, 85.60, 79.50, 79.13, 56.67, 55.99, 54.72, 54.23, 41.39, 40.81, 28.59, 28.21, 27.99, 20.33, 14.06, 11.38, 10.43, 8.33 ppm. IR (neat): νₘₐₓ 2977, 2930, 2870, 1752, 1692, 1456, 1392, 1367, 1350, 1285, 1244, 1152, 1105, 1057, 893, 847, 824, 756, 733. HRMS (ESI) m/z calculated for C₃₂H₃₄BrF₂N₃O₄ [M+H]⁺: clcd 642.1779, found 642.1784.

### Example 6: (1R,3S,4S)-tert-butyl 3-(5-(7-(1-(tert-butoxycarbonyl)-2-((S)-5-(tert-butoxycarbonyl)-5-azaspiro [2.4]heptan-6-yl)-1H-imidazol-5-yl)-9,9-difluoro-9H-fluoren-2-yl)-1H-benzo[d]imidazol-2-yl)-2-azabicyclo[2.2.1]heptane-2-carboxylate (15); (compound of Formula (X))

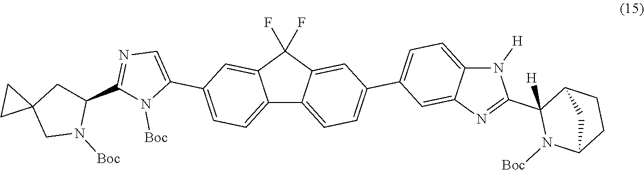 [SMILES: CC(C)(C)OC(=O)N1CC2(CC2)C[C@H]1c1ncc(-c2ccc3c(c2)C(F)(F)c2cc(-c4ccc5[nH]c([C@@H]6[C@H]7CC[C@H](C7)N6C(=O)OC(C)(C)C)nc5c4)ccc2-3)n1C(=O)OC(C)(C)C]

A 25 mL round-bottomed flask was charged with compound of Formula (13) (0.1 g), and compound of Formula (14) (0.08 g), Pd(OAc)₂ (0.0022 g), and PPh₃ (0.0045 g) and added DME (2 mL) followed by NaHCO₃ aqueous solution (1M, 0.62 mL). The reaction mixture was purged with N₂ gas and heated to 90° C. for 2 h under N₂. The reaction was cooled to rt and quenched with saturated NaHCO₃ aqueous solution. The mixture was extracted with EtOAc twice. The combined organic layer was washed with brine and dried over anhydrous sodium sulfate. Then the solvent was concentrated, and the residue was purified by using silica gel column chromatography to give the product of Formula (15) (0.11 g, 81%). The synthesis procedure is depicted in FIG. 4.

¹H NMR (500 MHz, CDCl₃): δ=10.72 (brd, J=26.18 Hz, 1H), 8.04 (d, J=8.58 Hz, 1H), 7.99 (s, 1H), 7.96-7.86 (m, 2H), 7.86-7.43 (m, 7H), rotamer; 5.62 (d, J=4.45 Hz, 0.5H), 5.60 (d, J=4.45 Hz, 0.5H), 4.59 (s, 1H), 4.18 (brs, 1H), 3.77 (d, J=10.14 Hz, 0.5H), 3.61 (d, J=10.14 Hz, 0.5H), 3.59-3.44 (m, 2H), 2.54 (dd, J=12.64, 7.98 Hz, 0.5H), 2.35 (dd, J=12.64, 7.98 Hz, 0.5H), 2.14-1.97 (m, 2H), 1.95-1.86 (m, 1H), 1.83-1.68 (m, 4H), 1.66 (s, 5H), 1.63 (s, 4H), 1.54 (s, 9H), 1.50 (s, 4H), 1.23 (s, 5H), 0.76-0.44 (m, 4H) ppm; ¹³C NMR (100 MHz, CDCl₃): δ=157.06, 155.32, 154.55, 154.08, 153.21, 151.71, 147.70, 147.61, 142.64, 142.55, 139.30, 139.00, 138.80, 138.57, 138.49, 138.32, 138.28, 137.97, 137.84, 134.37, 133.79, 131.06, 129.19, 128.87, 123.12, 122.71, 120.77, 120.67, 120.59, 120.39, 113.82, 113.04, 85.67, 85.49, 80.85, 79.51, 79.16, 61.50, 58.50, 56.69, 56.00, 54.73, 54.25, 41.40, 40.82, 36.99, 30.39, 28.60, 28.00, 26.89, 20.04, 14.08, 11.32, 10.49, 8.34 ppm. IR (neat): νₘₐₓ 2975, 2930, 2873, 1752, 1686, 1454, 1392, 1366, 1286, 1253, 1153, 1104, 1049, 908, 837, 814, 753, 730, 664, 647 cm⁻¹. HRMS (ESI) m/z calculated for C₃₂H₃₄BrF₂N₃O₄ [M+H]⁺: clcd 875.43460, found 875.43022.

### Example 7: (S)-tert-butyl 5-(7-bromo-9,9-difluoro-9H-fluoren-2-yl)-2-(1-(tert-butoxycarbonyl)-4-methylenepyrrolidin-2-yl)-1H-imidazole-1-carboxylate (16); (compound of Formula (VIII))

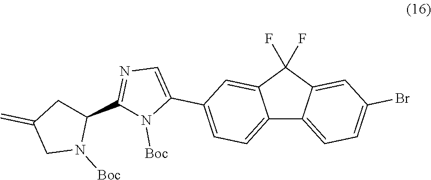 [SMILES: C=C1C[C@@H](c2ncc(-c3ccc4c(c3)C(F)(F)c3cc(Br)ccc3-4)n2C(=O)OC(C)(C)C)N(C(=O)OC(C)(C)C)C1]

The compound of Formula (11) (0.05 g) and N-fluorobenzenesulfonimide (0.079 g) were taken into a round bottom flask and added THF. The solution was degassed three times by slowly applying vacuum, followed by breaking the vacuum with nitrogen. The solution was cooled to −78° C. A solution of the base (1.0M LiHMDS in THF 0.25 mL) was added at such a rate the internal temperature was kept below −55° C. The progress of the reaction was monitored by TLC. After completion of the reaction, it was quenched by the addition of methanol. After the internal temperature had warmed to −20° C. to 0° C., hexane was added, and the solution was stirred for 10 minutes. The slurry was filtered, and the solids were rinsed with EtOAc twice. The filtrate was concentrated under reduced pressure to give the product of Formula (16) (0.036 g, 70%).

¹H NMR (500 MHz, CDCl₃): δ=8.00 (s, 1H), 7.93 (s, 0.5H), 7.86 (d, J=7.81 Hz, 0.5H), 7.74 (s, 1H), 7.63-7.57 (m, 2H), 7.52 (t, J=7.50 Hz, 1H), 7.42 (t, J=8.10 Hz, 1H), 5.62 (d, J=8.58 Hz, 0.5H), 5.58 (d, J=8.58 Hz, 0.5H), rotamer; 5.05 (s, 0.5H), 4.99 (s, 0.5H), 4.94 (d, J=18.10 Hz, 1H), 4.41 (t, J=17.69, 1H), 4.26-4.18 (m, 1H), 3.21-3.11 (brm, 1H), 2.66 (d, J=16.01 Hz, 0.5H), 2.6 (d, J=16.01 Hz, 0.5H), 1.66 (s, 5H), 1.64 (s, 4H), 1.51 (s, 5H), 1.24 (S, 4H) ppm. ¹³C NMR (100 MHz, CDCl₃): 154.79, 154.31, 152.52, 152.36, 151.10, 150.98, 147.66, 147.56, 147.49, 144.49, 143.52, 143.16, 143.00, 139.80, 139.19, 138.74, 138.44, 135.04, 133.96, 133.27, 129.26, 129.19, 128.92, 128.01, 127.61, 127.27, 122.87, 122.71, 121.70, 121.62, 121.42, 121.37, 120.85, 120.78, 120.54, 120.43, 120.28, 114.24, 113.89, 113.51, 113.16, 107.74, 107.56, 92.75, 91.01, 85.96, 85.82, 85.69, 79.85, 79.47, 56.18, 55.88, 51.42, 51.23, 39.10, 38.78, 28.57, 28.41, 27.99 ppm. IR (neat): νₘₐₓ 2978, 2929, 2855, 1699, 1392, 1368, 1286, 1252, 1153, 1106, 897, 845, 772, 733 cm-1. HRMS (ESI) m/z calculated for C₃₁H₃₃BrF₂N₃O₄ [M+H]⁺: 628.1627, found: 628.1623.

### Example 8: (S)-tert-butyl 6-(5-(7-bromo-9,9-difluoro-9H-fluoren-2-yl)-1-(tert-butoxycarbonyl)-1H-imidazol-2-yl)-5-azaspiro[2.4]heptane-5-carboxylate (13); (compound of Formula (IX))

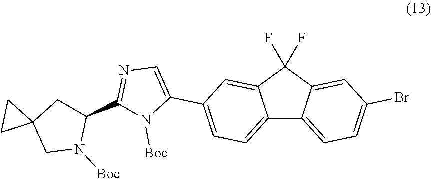 [SMILES: CC(C)(C)OC(=O)N1CC2(CC2)C[C@H]1c1ncc(-c2ccc3c(c2)C(F)(F)c2cc(Br)ccc2-3)n1C(=O)OC(C)(C)C]

Diethylzinc (1 M in hexane, 0.478 mL) was added to a stirred solution of the compound of Formula (16) (0.05 g) in an anhydrous DCM at 0° C. Diiodomethane (0.077 mL) was added slowly, keeping the internal temperature below 5° C. After addition completed, the mixture was stirred at 0° C. for 1 hr. The reaction was quenched with a saturated aqueous NH₄Cl solution and water. The layers were separated, and the aqueous layer was extracted with DCM. The combined organic layers were dried over anhydrous sodium sulfate and concentrated under reduced pressure to give the product of Formula (13) (0.036 g, 72%).

### Example 9: (S)-tert-butyl 6-(1-(tert-butoxycarbonyl)-5-(9,9-difluoro-7-(4,4,5,5-tetramethyl-1,3-dioxolan-2-yl)-9H-fluoren-2-yl)-1H-pyrrol-2-yl)-5-azaspiro[2.4]heptane-5-carboxylate (17); (compound of Formula (XI))

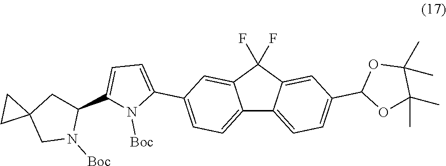 [SMILES: CC(C)(C)OC(=O)N1CC2(CC2)C[C@H]1c1ccc(-c2ccc3c(c2)C(F)(F)c2cc(C4OC(C)(C)C(C)(C)O4)ccc2-3)n1C(=O)OC(C)(C)C]

The compound of Formula (13) (0.05 g), Bis(pinacolato)diborane (0.039 g) and potassium acetate (0.019 g) were suspended in 1,4-dioxane (1 Ml) and stirred under vacuum for 5 minutes. PdCl₂(dppf)₂ (0.0028 g) was added and the reaction mixture was heated at 90° C. for 1 hr. After completion of the reaction, it was brought to rt, and the solvents were evaporated under reduced pressure. The crude residue was dissolved in EtOAc and washed with brine. The organic layer was dried over anhydrous sodium sulfate and concentrated under reduced pressure to give the product of Formula (17) (0.039 g, 74%) was isolated as a white solid.

¹H NMR (500 MHz, DMSO-d₆) δ=8.23-8.12 (m, 2H), 8.15-8.06 (m, 1H), 7.94-7.85 (m, 4H), 5.51-5.46 (m, 0.5H), 5.43-5.39 (m, 0.5H), 3.59-3.37 (m, 2H), 1.96-1.80 (m, 1H), 1.76-1.70 (m, 1H), 1.61 (s, 5H), 1.41 (s, 4H), 1.33 (s, 9H), 1.17 (s, 12H), 0.65-0.44 (m, 4H) ppm.

### Example 10: (1R,3S,4S)-tert-butyl 3-(5-(7-(1-(tert-butoxycarbonyl)-2-((S)-5-(tert-butoxycarbonyl)-5-azaspiro [2.4]heptan-6-yl)-1H-imidazol-5-yl)-9,9-difluoro-9H-fluoren-2-yl)-1H-benzo[d]imidazol-2-yl)-2-azabicyclo[2.2.1]heptane-2-carboxylate (15); (compound of Formula (I))

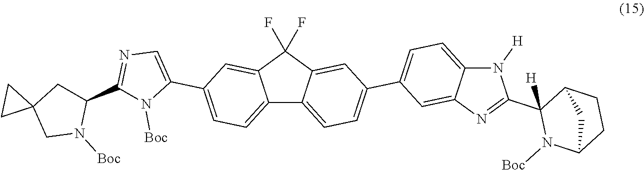 [SMILES: CC(C)(C)OC(=O)N1CC2(CC2)C[C@H]1c1ncc(-c2ccc3c(c2)C(F)(F)c2cc(-c4ccc5[nH]c([C@@H]6[C@H]7CC[C@H](C7)N6C(=O)OC(C)(C)C)nc5c4)ccc2-3)n1C(=O)OC(C)(C)C]

A 25 mL round bottomed flask was charged with compound of Formula (17) (0.1 g), and bromo compound of formula (18) (0.08 g), pd(OAc)₂ (0.0022 g), and pph₃ (0.0045 g). DME was added followed by NaHCO₃ aqueous solution (1M, 0.62 mL). The reaction mixture was purged with N₂ and heated at 90° C. for 2 h under N₂. The reaction was cooled to rt and quenched with saturated NaHCO₃ aqueous solution. The mixture was extracted with EtOAc twice. The combined organic layer was washed with brine and dried over anhydrous sodium sulphate, filtration concentration, and purification by silica gel chromatography. The product 15 (0.11 g, 81%) was isolated as yellowish foam.

### Advantages of the Invention

The various advantages of the present process are given below:
1. Reduced number of steps;
2. Improved yields of the product with late stage cyclopropanation;
3. Minimal usage of expensive fluorinating agent due to the late-stage fluorination; and
4. An easy operational process which provides improved overall yield of the product.

We claim:

1. A process for preparation of Ledipasvir compound of Formula I, its pharmaceutically acceptable salt or solvate thereof, 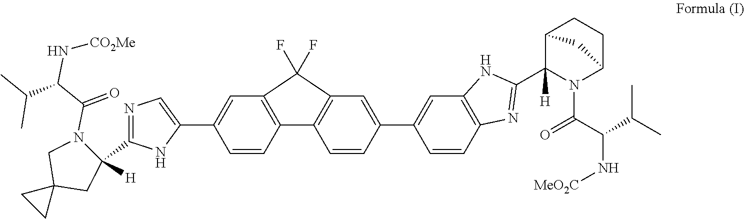 [SMILES: COC(=O)N[C@H](C(=O)N1CC2(CC2)C[C@H]1c1ncc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)[nH]1)C(C)C]
comprising the steps of:
i) coupling a compound of formula (II) or its reactive derivative with a compound of formula (III) in the presence of a solvent and optionally using a catalyst to give a compound of formula (IV); 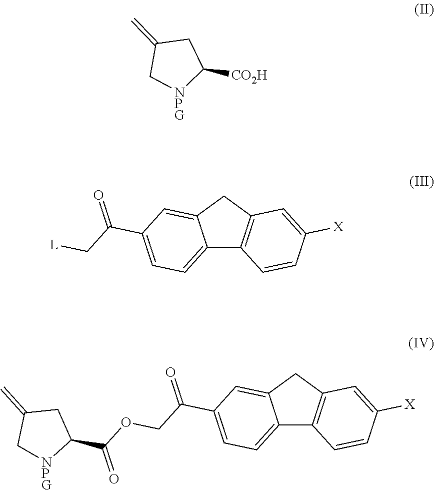
wherein
PG is a protecting group selected from the group consisting of Carbobenzyloxy (Cbz), tert-Butyloxycarbonyl (Boc), p-Methoxy carbonyl (Moz or MeOZ), 9-Fluorenylmethyloxycarbonyl (FMOC), Acetyl (Ac), Benzoyl (Bz), Benzyl carbamate, Tosyl (Ts), and Sulfonamides;
L is a leaving group selected from the group consisting of halogens, in particular Cl, Br, F or I; alkyl boronate esters, cycloalkyl boronate esters, mesyloxy, acyloxy, tosyloxy, benzyloxy, trifluoromethylsulfonyloxy, nonafluoraobutylsulfonyloxy, (4-bromo-phenyl)sulfonyloxy, (4-nitro-phenyl)sulfonyloxy, (2-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutylphenyl)sulfonyloxy, and (4-methoxy-phenyl)sulfonyloxy, preferably Cl;
X is halo or (RO)₂B—;
R is C₁₋₆ alkyl, aryl or R groups combined together to form substituted cycloalkyl group;
ii) cyclization of the compound of formula (IV) as obtained in step (i) using a cyclizing agent in a solvent to obtain a compound of formula (V); 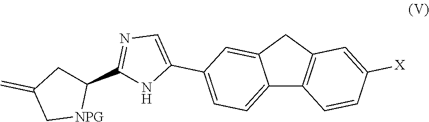
iii) protecting the compound of formula of (V) as obtained in step (ii) with a protecting group [PG] and in a solvent to give a compound of formula (VI); 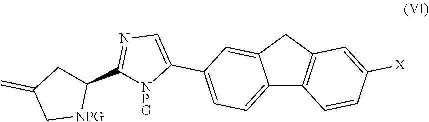
iv) cyclopropanation on the compound of Formula (VI) as obtained in step (iii) to give a compound of Formula (VII) followed by fluorination on the compound of Formula (VII) to give a compound of Formula (IX), or fluorination on the compound of Formula (VI) to give a compound of Formula (VIII) followed by cyclopropanation on the compound of Formula (VIII) using a reagent and in a first solvent to give a compound of Formula of (IX); 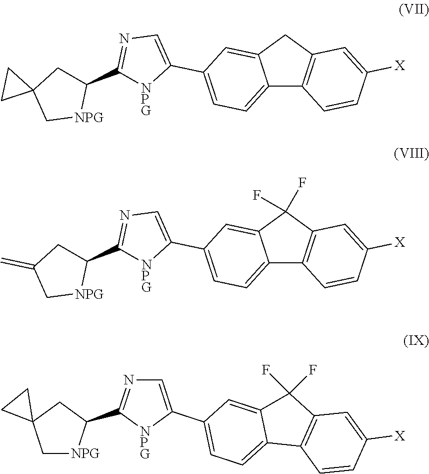
v) reacting the compound of Formula (IX) as obtained in step (iv) or its salts with a compound of Formula (IXa) using a metal catalyst and a base in the presence of a solvent to give a compound of Formula (X) 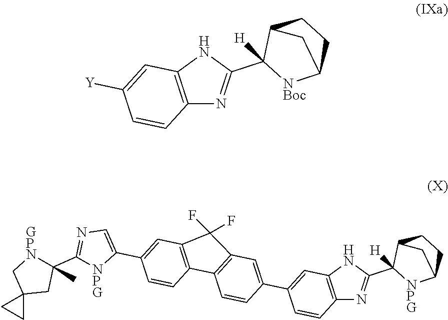
wherein
when X is halo in Formula IX, Y is —B(OR)₂ in Formula IXa;
vi) converting compound of Formula (IX) as obtained in step (iv) or its salts with a boronate ester using a metal catalyst and a base in the presence of a solvent to give a compound of Formula (XI); 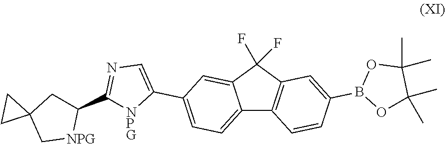
wherein
X is —B(OR)₂ in Formula IX, R is C₁₋₆ alkyl, aryl or R groups combined together to form substituted cycloalkyl group.
vii) alternatively, reacting the compound of Formula (XI) as obtained in step (vi) or its salts with the compound of Formula (XIa) using a metal catalyst and a base in the presence of a solvent to give a compound of Formula (X); 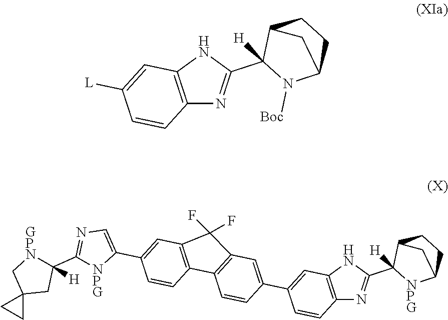
wherein Y is halo (Formula of IXa);
viii) removing protecting groups of the compound of Formula (X) as obtained in step (v) or step (vii) using an acid in a solvent followed by reaction with compound of a Formula (Xa) or its reactive derivative in the presence of a condensing agent and a solvent to give Ledipasvir of Formula I.

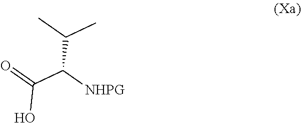

2. The process as claimed in claim 1, wherein the cyclizing agent is selected from the group consisting of ammonium acetate, K₂CO₃, sodium bicarbonate, or sodium bicarbonate.

3. The process as claimed in claim 1, wherein in step (iv), cyclopropanation is carried out in the presence of a sulfonium halide in the presence of a base, diiodomethane in the presence of diethylzinc, and a reagent selected from CF₃CO₂ZnCH₂I, or (PhO)₂P(O)ZnCH₂I, and preferably diiodomethane in the presence of diethylzinc.

4. The process as claimed in claim 1, wherein in step (iv), the fluorination is carried out using a fluorinating agent selected from the group consisting of 1-chloromethyl-4-fluoro-1, 4-diazoniabicyclo [2.2.2]octanebis(tetrafluoroborate), N-fluorobenzenesulfonimide, xenon fluorides, cobalt fluoride, silver(II) fluoride, tetraethylammonium tetrafluoroborate, hydrogen fluoride pyridine complex, potassium hydrogen fluoride, triethylamine trihydrofluoride, or 4-iodotoluene difluoride and preferably, N-fluorobenzenesulfonimide.

5. The process as claimed in claim 1, wherein the protecting group is selected from the group consisting of carbobenzyloxy (Cbz), tert-butyloxycarbonyl (Boc), p-methoxy carbonyl (Moz or MeOZ), 9-fluorenylmethyloxycarbonyl (FMOC), acetyl (Ac), benzoyl (Bz), benzyl carbamate, tosyl (Ts) and sulphonamides.

6. The process as claimed in claim 1, wherein the boronate ester is prepared using boronate reagent selected from the group consisting of pinacolboronates, alkyl boronates and aryl boronates.

7. The process as claimed in claim 1, wherein the metal catalyst is selected from the group consisting of Palladium(0) or (II) complexes, selected from the group consisting of tetrakis(triphenylphosphine)palladium, tris(dibenzylideneacetone)dipalladium, palladium dppf chloride, Bis(triphenylphosphine)palladium(II) acetate, and Bis(triethylphosphine)palladium(II) chloride).

8. The process as claimed in claim 1, wherein the base is selected from the group consisting of alkyl amines, NH₃, K₂CO₃, Na₂CO₃, NaHCO₃, NH₄OH, Mg(OH)₂, CaCO₃, Ca(OH)₂, KOH, NaOH, NaH, KH, KOtBu, CH₃CO₂Na, CH₃CO₂K, (CH₃)₃CONa, LiOH, N-Methylmorpholine, or mixtures thereof.

9. The process as claimed in claim 1, wherein the solvent is selected from water, dichloromethane (DCM), 1,2-dichloroethane (DCE), tetrahydrofuran (THF), alcohol selected from the group consisting of methanol, ethanol, n-propanol, isopropanol, n-butanol and t-butanol, hydrocarbon solvent selected from the group consisting of benzene, toluene, xylene, heptane, hexane, and cyclohexane, ketone solvents selected from the group consisting of acetone, ethyl methyl ketone, diethyl ketone, methyl tert-butyl ketone, and isopropyl ketone, ester solvents selected from the group consisting of methyl acetate, ethyl acetate, propyl acetate, isopropyl acetate, n-butyl acetate, isobutyl acetate, and sec-butyl acetate, nitrile solvents selected from the group consisting of acetonitrile, propionitrile, butyronitrile and isobutyronitrile, ether solvents selected from the group consisting of di-tert-butylether, dimethylether, diethylether, diisopropylether, 1,4-dioxane, methyl tert-butylether, ethyl tert-butylether, tetrahydrofuran, 2-methyl tetrahydrofuran, 2-methoxyethanol and dimethoxyethane, or amide solvents selected from the group consisting of formamide, dimethyl formamide (DMF), dimethylacetamide (DMAC), N-methyl-2-pyrrolidone, N-methylformamide, 2-pyrrolidone, 1-ethenyl-2-pyrrolidone, preferably selected from dichloromethane (DCM), toluene, acetone, 1,2-dichloroethane (DCE) and tetrahydrofuran (THF).

10. The process as claimed in claim 1, wherein the condensing agent used is selected from the group consisting of Benzotriazol-1-yl)-N,N,N,N-Hydroxy benzotriazole (HOBt), O-Tetramethyluronium fluorophosphate (HBTU), O-(Benzotriazol-1-yl)-N,N,N′,N′-tetramethyluroniumtetra fluoroborate (TBTU), 1-Hydroxy-7-azabenzotriazole (HOAt), N,N′-Dicyclohexyl carbodiimide (DCC), N-(3-Dimethylaminopropyl)-N′-ethyl carbodiimide hydrochloride (EDC-HCl), N,N′-Carbonyl diimidazole (CDI), Benzotriazol-1 yloxytris (dimethyl amino)phosphonium hexafluorophosphate (BOP), Benzotriazolyloxytripyrrolidinophosphonium hexafluorophosphate (PyBOP), and 1[Bis(dimethylamino)methylene]-1H-1,2,3-triazole[4,5-b]pyridinium 3-oxide hexafluorophosphate (HATU), or mixtures thereof.

11. A compound of Formula A 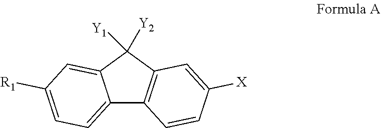
wherein R₁= 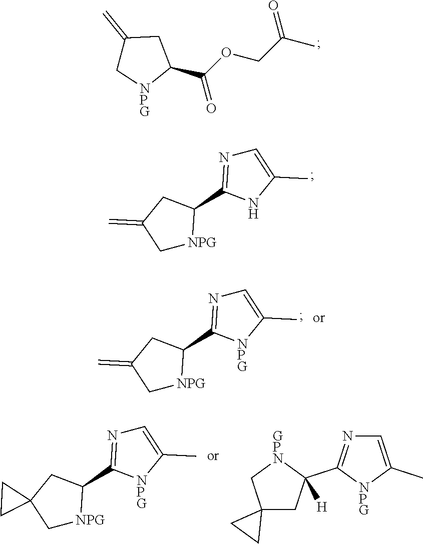
Y₁ and Y₂=H or F;
X is halo, (RO)₂B— or

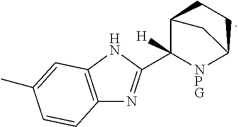

12. The compound as claimed in claim 11, wherein the compound of formula A is selected from the group consisting of: [chemical structure]
